(19) United States
(12) Patent Application Publication
(10) Pub. No.: US 2025/0143599 A1
(43) Pub. Date: May 8, 2025

(54) SYSTEMS AND METHODS FOR DETECTING LOCATIONS OF SUBCUTANEOUS TISSUE STRUCTURES

(71) Applicant: University of Notre Dame du Lac, South Bend, IN (US)

(72) Inventors: Thomas O'Sullivan, South Bend, IN (US); Patrick Fay, South Bend, IN (US); Sunghoon Rho, South Bend, IN (US)

(21) Appl. No.: 18/730,666

(22) PCT Filed: Jan. 20, 2023
(86) PCT No.: PCT/US2023/060992

§ 371 Date: Jul. 19, 2024

Related U.S. Application Data
(60) Provisional application No. 63/301,307, filed on Jan. 20, 2022.

(51) Int. Cl. A61B 5/06; A61B 5/00

(52) U.S. Cl. CPC A61B 5/065; A61B 5/7405; A61B 2560/0219

(57) ABSTRACT

A system for localizing a lesion includes an implant and a probe. The implant includes a power source; a light source; a matching network comprising circuitry to provide an amount of power from the power source to the light source; and a printed circuit board. The probe includes a radio-frequency source configured to transmit wireless power to the power source of the implant. The implant is proximate the lesion, and the light source is configured to produce a first light in response to the probe being a first distance from the implant and a second light in response to the probe being a second distance from the implant.

### CROSS-REFERENCE TO RELATED APPLICATIONS

This application is a non-provisional application claiming priority from U.S. Provisional Application No. 63/301,307 filed on Jan. 20, 2022 and entitled “OPTICALLY-ENHANCED WIRE-FREE BREAST LESION LOCALIZATION SYSTEM,” which is hereby incorporated by reference in its entirety.

### STATEMENT REGARDING FEDERALLY SPONSORED RESEARCH OR DEVELOPMENT

This invention was made with partial support by the National Institutes of Health under contract TR002529. The government has certain rights in the invention.

### TECHNICAL FIELD

This disclosure generally relates to systems of methods for detecting a location of an implant. Particularly, in one example this disclosure relates to detecting the location of an implant based on a light on the implant that is powered by an external signal.

### BACKGROUND

It is estimated that approximately 268,000 lumpectomy procedures are performed each year in the United States, of which 25-35% are for nonpalpable breast lesions. Surgical removal of these nonpalpable lesions typically requires the use of localization techniques to guide the surgeon during lumpectomy. The most common technique in use today is wire localization (WL), which entails the preoperative implantation of 3 to 15 cm wires under imaging guidance to bracket the lesion location. The distal end of the wire contains a tissue anchor (e.g., a hook or barb), while the proximal end of the wire protrudes from the skin. By following the length of the wire, the surgeon can identify the tissue volume to be excised.

WL procedures oftentimes have significant drawbacks: wire must be implanted the day of surgery—typically by Radiology—and patient movement is limited once placed. Besides obvious patient discomfort and scheduling challenges, WL complications also include displacement/migration of the wire due to forces on the external component and retention of wire fragments.

To overcome these limitations, several “wire-free” localization devices have been recently developed based on different modalities (radioactive, magnetic, radio-frequency). These approaches involve the preoperative image-guided placement of a small, millimeter-sized implant that can be noninvasively detected utilizing an external handheld sensing probe. For example, there is a commercially-available 12×1 mm infrared light powered radar reflector implant that can be detected up to 60 mm deep with a handheld probe. The probe and console unit generates an audio tone of increasing frequency as the probe gets closer to the implant and displays the distance between the two with 1 mm accuracy. Importantly, it can be implanted more than 30 days before surgery, greatly simplifying scheduling and convenience.

However, this implant device and similar wire-free devices also may have several drawbacks. First, with existing technology, it is difficult to localize multiple devices in the breast closer than 20-25 mm. This is because, even though linear distance between the probe and implant can be measured with 1 mm accuracy, the signal is widely perceivable from all directions (360°) relative the implant. This prevents the use of multiple devices to bracket smaller areas of suspicion. Second, because breast surgeons typically choose to create a breast incision in an inconspicuous location to improve cosmetic outcome, surgeons oftentimes excise tissue through a “tunnel” rather than over top of the lesion. This can make precise 3D localization of the implant with the probe difficult. With its 12 mm long antennae, the device is also rather large for use with small lesions.

### BRIEF DESCRIPTION OF THE DRAWINGS

FIG. 1 is a diagram of an example system for localizing a lesion in accordance with the various examples disclosed herein.

FIG. 2 is a diagram of the implant of the system of FIG. 1 in accordance with the various examples disclosed herein.

FIG. 3 is a diagram of the implant and of a probe of the system of FIG. 1 in accordance with the various examples disclosed herein.

FIG. 4 is a flow chart illustrating an example method of deter localizing a lesion in accordance with the various examples disclosed herein.

### DETAILED DESCRIPTION

The following disclosure of example methods and apparatus is not intended to limit the scope of the disclosure to the precise form or forms detailed herein. Instead, the following is intended to be illustrative so that others may follow its teachings.

The challenge presented here is to provide a localization system that is as accurate for a medical professional and as comfortable for a patient as possible. As described above, current localizations systems often fail one or both of these criteria, as wire-based localization systems are usually uncomfortable if not outright painful for patients while simultaneously being prone to accidents, and current wire-free solutions oftentimes sacrifice accuracy and the ability to locate multiple probes in a single patient. Therefore, these multi-faceted challenges necessitate improving the system and method by which medical professionals determine the location of an implant within a patient.

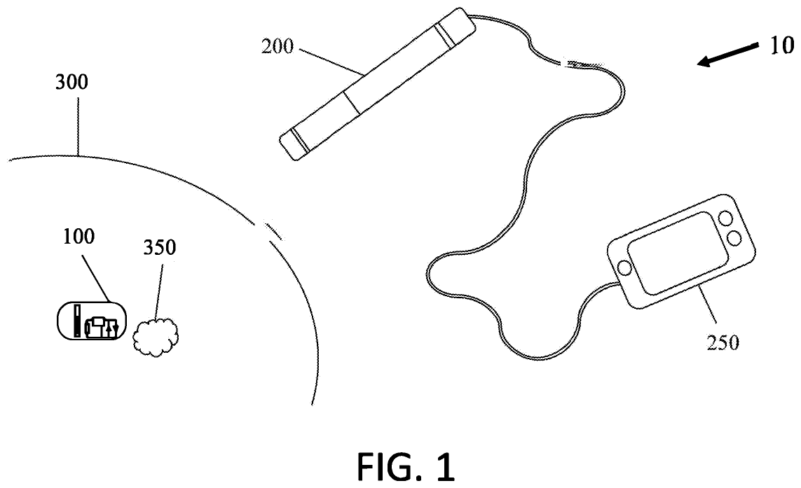

FIG. 1 is a diagram of an example system 10 utilized for localizing a subcutaneous tissue structure (e.g., lesion, tumor, etc.) within a patient. As shown in FIG. 1, the example system 10 includes an implant 100, a probe 200, a user device 250, a patient 300, and a lesion 350. In this example, the probe 200 provides power to the implant 100, which illuminates one or more lights on the implant 100 in response to the received power. The quality of the light (e.g., color, brightness, etc.) is affected by the amount of provided power, such that a first quality of light (e.g., dim light) can be indicative of a low amount of power and a second quality of light (e.g., bright light) can be indicative of a high amount of power. Because the amount of power provided by the probe 200 is inversely proportional to a distance between the probe 200 and the implant 100, the quality of the light from the implant 100 can indicate a distance between the probe 200 and the implant 100.

Furthermore, due to the natural light absorption properties of tissue, the color of the lights on the implant 100 may be perceived by a user as changing based on a depth of the implant. For example, the wavelength of red light is not well-absorbed by tissue, such that a white light shown through tissue may appear as red because the other wavelengths of light in the white light are absorbed while the red light is not. Because the wavelength of blue light is well-absorbed by tissue, if a white light shown through tissue appears as blue (e.g., purple, in combination with the red light), the tissue must not be thick. Therefore, if the implant 100 is providing a white light, the implant 100 light appears as red if deep within the tissue and as purple if closer to the surface of the tissue. This change in color may indicate to the user that they are getting close to the implant (e.g., in cutting away the correct tissue).

The user device 250 is in connection with the probe 200, and is configured to control one or more aspects of the probe 200, provide power to the probe 200, to display information from the probe 200, and/or provide any other suitable connection communication. For example, in those examples in which the probe 200 is configured to calculate a distance from the probe 200 to the implant 100, the user device 250 displays this determined distance on a display of the user device 250. Although the connection between the user device 250 and the probe 200 is shown to be wired, this connection may be, in some examples, wireless (e.g., via Bluetooth™, etc.)

In this example, the lesion 350 is a breast lesion (such that the portion of the patient 300 shown in FIG. 1 is the patient's breast), which is a portion or area of abnormal tissue, and is the target of a lumpectomy procedure. It will be understood that the lesion 350 may be any suitable lesion or other tissue as desired. A position of the lesion 350 is determined using one or more existing methods of detection (e.g., mammogram, X-ray, MRI, etc.), and the implant 100 is implanted into the patient 300 based on the detected location. Advantageously, and in contrast to current wire-based systems, this lesion-detection and implant process may be performed well in advance (e.g., day(s) prior) of the corresponding lumpectomy, but need not need be.

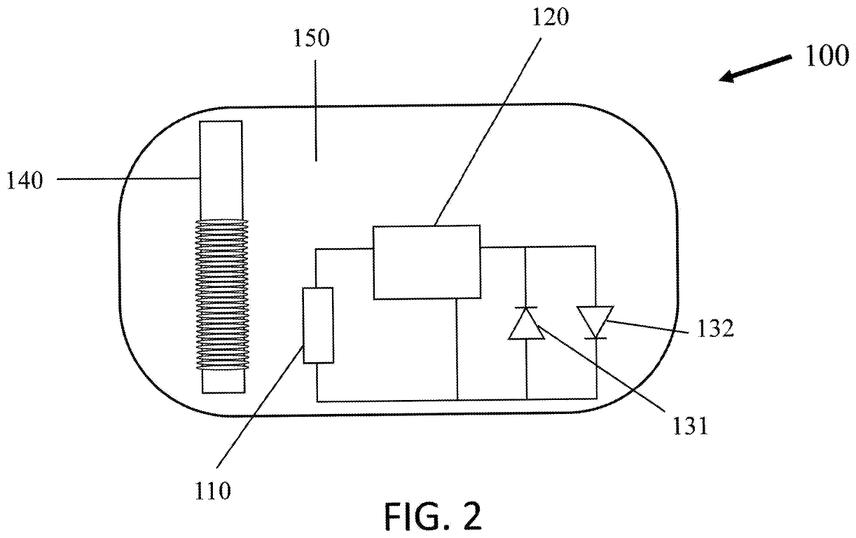

FIG. 2 is a diagram of an example configuration of the implant 100. As shown in FIG. 2, the implant 100 includes a power source 110, a matching network 120, a first light 131 and a second light 132 (collectively “lights 130”). Each of these components is positioned on a printed circuit board 150. In this example in which the implant 100 is powered by inductively coupling with the probe 200, the power source 110 may include an antenna 140 formed as a coil. In other examples, the power source 110 may be a solar cell capable of being charged by infrared light, may be powered by ultrasound waves, or may be a battery. The antenna 140 may be a 13.56 MHz inductive coil, 2.4 GHz dipole antenna, or other reasonable frequency, and the design of the antenna 140 is configured to resonate with a signal (e.g., a radio-frequency signal from the probe 200), which generates power for the power source 110. The matching network 120 is configured to transfer the sinusoidal signal from the power source 110 to the lights 130 while losing as little power as possible. In this example, the circuitry of the matching network 120 is a single series capacitor due to the configuration of the antenna 140, but other configurations for the matching network 120 are contemplated and should be interpreted as within the scope of this disclosure. For example, a near-field communication (NFC)-integrated circuit may be used in the matching network 120.

In this example configuration, the amount of power required to illuminate the lights 130 (e.g., 1 mW) can be provided with a 5 W signal from the probe 200 from 50 cm away. This 5 W power is well below the threshold for patient safety and is roughly equal to the radiated power of a typical handheld radio.

The lights 130 may be any suitable light source capable of providing light at relatively low amounts of power, such as light-emitting diodes (LEDs) and/or diode lasers, and may be powered by any suitable power source as desired. In this example, the first and second lights 131, 132 are both LEDs and are biased in opposite directions (e.g., in anti-parallel), with the first light 131 providing a first color (e.g., red) and the second light 132 providing a second color (e.g., blue). Because the lights 130 are arranged in anti-parallel, the bias of the first light 131 is always opposite of the bias of the second light 132. The load impedance here may be chosen as the average impedance of both possible configurations (e.g., first light 131 forward/second light 132 reverse and first light 131 reverse/second light 132 forward). The matching network 120 is selected to match this load impedance to that of the power source 110. In one example, the average impedance of the lights 130 in the first configuration (e.g., first light 131 forward/second light 132 reverse) is about 1.65V, and the average impedance of the lights 130 in the second configuration (e.g., first light 131 reverse/second light 132 forward) is about 2.45V, for an average of 2.05V. This impedance value enables the lights 130 to match (e.g., via the matching network 120) the voltage at the power source 110, which, in turn, enables maximum power transfer from the power source 110 to each of the lights 130 at different voltage levels.

Although the lights 130 are shown here as separate lights, in some examples, the lights 130 may be a single light configured to illuminate with multiple colors or brightness levels, such that the single light may provide the same multiple functionality with a single component. Further, in some examples, more than two lights may be included, as this disclosure should not be read as limited to only the two-light configuration shown in FIG. 2.

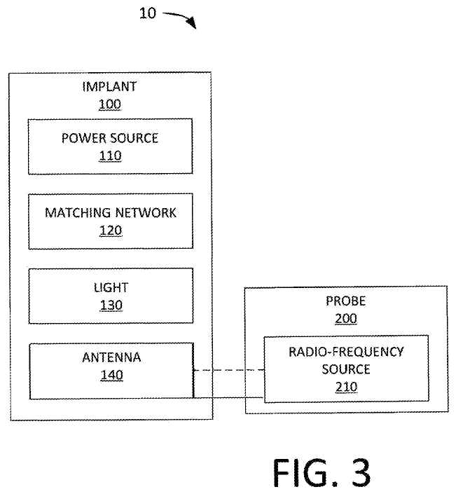

FIG. 3 is a diagram of the implant 100 and of the probe 200. As shown in FIG. 2, the probe 200 includes a radio-frequency source 210 configured to generate and transmit a radio-frequency signal that resonates with the antenna 140 of the implant 100. In this example, the radio-frequency source 210 transmits the signal in the industrial, scientific, and medical (ISM) band of 13.56 MHz.

In some examples, the system 10 determines (e.g., calculates) a distance from the probe 200 to the implant 100 separately from illuminating the lights 130. By monitoring the time-of-flight phase difference from passive reflection of the signal from the radio-frequency source 210, the distance traveled by the signal (which is twice the distance between the probe 200 and the implant 100), the system 10 can calculate the distance. This calculated distance is then displayed on the user device 250. Alternatively (or additionally), the user device 250 or probe 200 may produce a sound or tone in response to the probe 200 being within a threshold distance (e.g., 10 cm) of the implant, in order to set an initial starting point for the medical professional.

Advantageously, the example implant 100 is sized to fit within and be implanted by a relatively small needle (e.g., 11G, 12G, 16G, etc.). This size is accomplished via the simplicity of the components of the implant 100, such as the single capacitor that forms the matching network 120 and the shape and length of the antenna 140. In particular, prior to implantation, the antenna 140 is arranged parallel to a central axis of the implant 100 to reduce the profile size of the implant 100, but is spring-loaded to bias to a perpendicular (relative to the implant 100) position once implanted, which also causes the antenna 140 to function as a tissue anchor. Furthermore, the example implant 100 is encased in a cylindrical outer shell formed from a micropipette (e.g., of glass, silicone, epoxy, parylene, etc.) and covered in a biocompatible coating, which not only provides protection to the implant 100 components but also allows the implant 100 to fit within a needle. Finally, because this 12G needle is the most common size for breast biopsies, this means that the implant 100 can be used with what is essentially the standard size for these procedures.

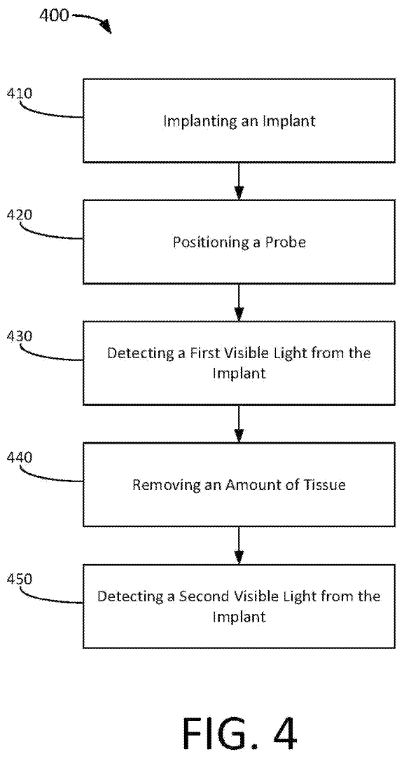

FIG. 4 is a flow chart of an example implementation of the system 10. As shown in FIG. 4, a method 400 for localizing a lesion 350 using an implant 100 begins with a step 410, where the implant 100 is implanted in a patient 300. In this example, the implant 100 is implanted proximate to a detected breast lesion 350, such that the implant is relied upon as a marker for the lesion 350. Because the implantation process requires only a needle, this step 410 is able to be performed in concert with traditional imaging methods, such that the location of the lesion 350 can be determined simultaneously with the implanting of the implant 100.

At a step 420, a probe 200 is positioned relative to the patient 300 and, at a step 430, a first visible light is detected from the implant 100. As described above, the light from the implant 100 is based on an amount of power received from the probe 200, which is itself based on a distance from the probe 200 to the implant 100. As such, this first visible light is indicative of the distance between the probe 200 to the implant 100. Put differently, one or more qualities of the first visible light indicate how far the probe 200 is from the implant 100.

At a step 440, an amount of tissue is removed from the patient 300 and, at a step 450, a second visible light is detected from the implant 100. Because the quality (e.g., color) of the light indicates a distance to the implant 100 (e.g., an amount of tissue between the implant 100 and ambient), a change in quality from the first light to the second light indicates whether the amount of removed tissue at step 440 brought the implant 100 closer to ambient. For example, if the first light is red while the second light is blue, or if the second light is brighter than the first light, the implant 100 is closer to the surface. Steps 440 and 450 may be repeated as necessary.

Some portions of the detailed descriptions of this disclosure have been presented in terms of procedures, logic blocks, processing, and other symbolic representations of operations on data bits within a computer or digital system memory. These disclosures and representations are the means used by those of ordinary skill in the art of data processing to most effectively convey the substance of their work to others of ordinary skill in the art. A procedure, logic block, process, etc., is herein, and generally, conceived to be a self-consistent sequence of steps or instructions leading to a desired result. The steps are those requiring physical manipulations of physical quantities. Usually, though not necessarily, these physical manipulations take the form of electrical or magnetic data capable of being stored, transferred, combined, compared, and otherwise manipulated in a computer system or similar electronic computing device. For reasons of convenience, and with reference to common usage, such data is referred to as bits, values, elements, symbols, characters, terms, numbers, or the like, with reference to various presently disclosed examples. It should be borne in mind, however, that these terms are to be interpreted as referencing physical manipulations and quantities and are merely convenient labels that should be interpreted further in view of terms commonly used in the art. Unless specifically stated otherwise, as apparent from the discussion herein, it is understood that throughout discussions of the present example, discussions utilizing terms such as “determining” or “outputting” or “transmitting” or “recording” or “locating” or “storing” or “displaying” or “receiving” or “recognizing” or “utilizing” or “generating” or “providing” or “accessing” or “checking” or “notifying” or “delivering” or the like, refer to the action and processes of a computer system, or similar electronic computing device, that manipulates and transforms data.

While this disclosure has described certain examples, it will be understood that the claims are not intended to be limited to these examples except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed examples. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

What is claimed is:

1. A system for localizing a lesion, the system comprising:
an implant comprising:
a power source;
a light source;
a matching network comprising circuitry to provide an amount of power from the power source to the light source; and
a printed circuit board; and
a probe comprising a radio-frequency source configured to transmit wireless power to the power source of the implant,
wherein the implant is proximate the lesion, and
wherein the light source is configured to produce a first light in response to the probe being a first distance from the implant and a second light in response to the probe being a second distance from the implant.

2. The system of claim 1, wherein the radio-frequency source transmits the wireless power in an industrial, scientific, and medical (ISM) band.

3. The system of claim 1, wherein the implant further comprises a biocompatible coating.

4. The system of claim 1, wherein the light source further comprises a first light-emitting diode (LED) and a second LED, and
wherein the first LED provides light of a first color and the second LED provides light of a second color.

5. The system of claim 1, wherein the first light comprises a first brightness, and the second light comprises a second brightness, the second brightness different than the first brightness.

6. The system of claim 1, wherein the implant is configured to emit a sound in response to a determination that the probe is within a threshold distance of the implant.

7. The system of claim 1, wherein:
the second distance is less than the first distance; and
the second light is brighter than the first light.

8. The system of claim 1, wherein:
the implant is configured to determine a distance between the implant and the probe as the first distance or the second distance based on an amount of wireless power received from the radio-frequency source, and
the amount of wireless power received from the radio-frequency source increases as the distance between the implant and the probe decreases.

9. The system of claim 1, wherein the implant is sized to be implanted via a 12G needle.

10. The system of claim 1, wherein the power source comprises a single-series capacitor.

11. The system of claim 1, wherein the power source comprises an antenna configured to receive the transmitted wireless power.

12. The system of claim 11, wherein the antenna comprises a spring-load configured to bias the antenna such that the antenna anchors the implant.

13. The system of claim 11, wherein the antenna is a 2.4 GHz half-wave dipole antenna.

14. A method of localizing a lesion, the method comprising:
implanting an implant into a portion of tissue proximate the lesion, the implant comprising:
a power source;
a light source;
a matching network, comprising circuitry to provide an amount of power from the power source to the light source; and
a printed circuit board;
detecting a first visible light from the implant by a probe, the probe comprising a radio-frequency source configured to transmit wireless power to the power source of the implant;
removing an amount of the portion of tissue; and
detecting a second visible light from the implant,
wherein the first visible light is in response to the implant being a first distance from a surface of the portion of tissue and a second visible light is in response to the implant being a second distance from the surface of the portion of tissue.

15. The method of claim 14, wherein the light source further comprises a first light-emitting diode (LED) and a second LED.

16. The method of claim 14, wherein the first visible light comprises a first color, and the second visible light comprises a second color, the second color different than the first color.

17. The method of claim 14, wherein:
the implant is configured to determine a distance between the implant and the probe as the first distance or the second distance based on an amount of wireless power received from the radio-frequency source, and
the amount of wireless power received from the radio-frequency source increases as the distance between the implant and the probe decreases.

18. An apparatus for localizing a lesion, the apparatus comprising:
an implant comprising:
a power source;
a light source; and
a matching network comprising circuitry to provide a first amount of power from the power source to the light source; and
a probe comprising a radio-frequency source configured to transmit a second amount power to the power source of the implant,
wherein:
the implant is proximate the lesion, and
the second amount of power increases as a distance between the probe and the implant decreases, and the first amount of power is directly proportional to the second amount of power.

19. The apparatus of claim 18, wherein the light source provides a first light in response to the first amount of power being low and a second light in response to the first amount of power being high.

20. The apparatus of claim 19, wherein the first light comprises a first color, and the second light comprises a second color, the second color different than the first color.
